(19) United States
(12) Patent Application Publication
(10) Pub. No.: US 2024/0238468 A1
(43) Pub. Date: Jul. 18, 2024

(54) SYSTEM FOR ASSISTING IN DISINFECTION ACTION, METHOD FOR ASSISTING IN DISINFECTION ACTION, SYSTEM FOR DETERMINING WHETHER DISINFECTION ACTION HAS BEEN PERFORMED, AND METHOD FOR DETERMINING WHETHER DISINFECTION ACTION HAS BEEN PERFORMED

(71) Applicant: CANON KABUSHIKI KAISHA, Tokyo (JP)

(72) Inventors: HIROSHI SAITO, Kanagawa (JP); MASARU SUGITA, Tokyo (JP); MASANORI SEKI, Kanagawa (JP); RYUICHI OTSU, Tokyo (JP)

(21) Appl. No.: 18/622,112

(22) Filed: Mar. 29, 2024

Related U.S. Application Data
(63) Continuation of application No. PCT/JP2022/036363, filed on Sep. 29, 2022.

(30) Foreign Application Priority Data
Oct. 1, 2021 (JP) 2021-162903
Oct. 1, 2021 (JP) 2021-162904

(51) Int. Cl. A61L 2/28

(52) U.S. Cl. CPC A61L 2/28; A61L 2202/14

(57) ABSTRACT

A system for assisting in a disinfection action in a disinfection target area, the system includes a determination unit configured to acquire information about a determination result about whether the disinfection action has been performed in the disinfection target area, and a storage unit configured to store the determination result that the disinfection action has been performed in the disinfection target area, time information about the performed disinfection action, and location information about the performed disinfection action in association with each other.

### CROSS-REFERENCE TO RELATED APPLICATIONS

This application is a Continuation of International Patent Application No. PCT/JP2022/036363, filed Sep. 29, 2022, which claims the benefit of Japanese Patent Applications No. 2021-162904, filed Oct. 1, 2021, and No. 2021-162903, filed Oct. 1, 2021, all of which are hereby incorporated by reference herein in their entirety.

### BACKGROUND OF THE INVENTION

### Field of the Invention

The present invention relates to a system for assisting in disinfection actions, a method for assisting in disinfection actions, a system for determining whether a disinfection action has been performed, and a method for determining whether a disinfection action has been performed.

### Background Art

As a measure against infectious diseases, the importance of disinfection is increasing. This leads to a demand for achieving a state where a disinfection action is performed efficiently and thoroughly without omissions in an environment including a disinfection target area.

Japanese Patent Application Laid-Open No. 2017-169613 discusses a mobile autonomous robot that detects a processing target substance with a sensor and automatically sprays hypochlorous acid for disinfection and deodorization. According to Japanese Patent Application Laid-Open No. 2017-169613, the robot sprays hypochlorous acid based on a stored map indicating locations of processing target substances.

### CITATION LIST

### Patent Literature

PTL 1: Japanese Patent Laid-Open No. 2017-169613

However, Japanese Patent Application Laid-Open No. 2017-169613 is silent on methods for performing disinfection efficiently and thoroughly without omissions in actual situations.

### SUMMARY OF THE INVENTION

The present invention is directed to providing a system capable of performing disinfection efficiently and thoroughly without omissions in actual situations.

According to an aspect of the present invention, a system for assisting in a disinfection action in a disinfection target area, the system includes a determination unit configured to acquire information about a determination result about whether the disinfection action has been performed in the disinfection target area, and a storage unit configured to store the determination result that the disinfection action has been performed in the disinfection target area, time information about the performed disinfection action, and location information about the performed disinfection action in association with each other.

According to another aspect of the present invention, a system includes a detection unit configured to detect environmental change in a disinfection target area and output information about the environmental change, and a determination unit configured to determine whether a disinfection action has been performed in the disinfection target area, based on the information about the environmental change, and output a result of the determination.

According to yet another aspect of the present invention, a system includes a detection unit configured to detect environmental change in a disinfection target area and output information about the environmental change, and a transmission unit configured to transmit information about whether a disinfection action has been performed in the disinfection target area, based on the information about the environmental change.

Further features of the present invention will become apparent from the following description of exemplary embodiments with reference to the attached drawings.

### BRIEF DESCRIPTION OF THE DRAWINGS

FIG. 1 is a block diagram illustrating a system according to a first exemplary embodiment of the present invention.

FIG. 2 is a flowchart illustrating a method according to the first exemplary embodiment of the present invention.

FIG. 3 is a diagram illustrating an example of a server apparatus according to the first exemplary embodiment of the present invention.

FIG. 4A is a diagram illustrating a disinfectant visualization sheet according to a second exemplary embodiment of the present invention.

FIG. 4B is a diagram illustrating a disinfectant visualization sheet according to the second exemplary embodiment of the present invention.

FIG. 4C is a diagram illustrating a disinfectant visualization sheet according to the second exemplary embodiment of the present invention.

FIG. 4D is a diagram illustrating a disinfectant visualization sheet according to the second exemplary embodiment of the present invention.

FIG. 5 is a diagram illustrating an example of a disinfection action report according to a third exemplary embodiment of the present invention.

FIG. 6 is a diagram illustrating an example of a disinfection action report according to the third exemplary embodiment of the present invention.

FIG. 7 is a flowchart illustrating a method according to a first modified example of the exemplary embodiments of the present invention.

FIG. 8A is a diagram illustrating a system according to third modified example of the exemplary embodiments of the present invention.

FIG. 8B is a diagram illustrating the system according to the third modified example of the exemplary embodiments of the present invention.

FIG. 9A is a diagram illustrating a system according to a fourth modified example of the exemplary embodiments of the present invention.

FIG. 9B is a diagram illustrating a system according to the fourth modified example of the exemplary embodiments of the present invention.

### DESCRIPTION OF THE EMBODIMENTS

Hereinafter, exemplary embodiments of the present invention will be described, but the present invention is not limited thereto.

### System

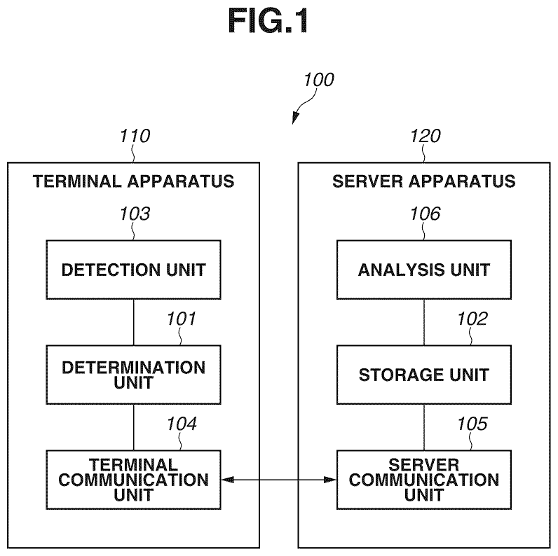

FIG. 1 is a diagram illustrating a system 100 according to exemplary embodiments of the present invention, and the system 100 is configured to assist in disinfection actions in disinfection target areas and includes at least a determination unit 101 and a storage unit 102. The determination unit 101 determines whether a disinfection action has been performed in the disinfection target areas and outputs the determination result. In a case where the determination unit 101 determines that a disinfection action has been performed in the disinfection target areas, the storage unit 102 stores the determination result output by the determination unit 101, location information about the disinfection target areas, and time information about the performed disinfection action in association with each other.

The system 100 according to the exemplary embodiments achieves efficient and thorough disinfection in actual situations without omissions. Specifically, in order to achieve a state where disinfection is performed thoroughly without omissions, information indicating that a disinfection action has been performed needs to be stored in the storage unit 102. With the information being stored in the storage unit 102, whether a disinfection action has been performed can be checked without relying on human memory, so that in a case where there is an omission of a disinfection action, the omission is detected. In order to achieve an efficient disinfection action, location and time information about the performed disinfection action also needs to be stored. In an environment with a plurality of disinfection target areas, in order to perform disinfection efficiently, a suitable order of disinfection actions needs to be selected. In a case where there is no change in a disinfection action target environment, disinfection may be performed in a predetermined order. However, in actual situations, there may be an initially unexpected obstacle, or a layout in the environment may be changed periodically. In such actual situations, in order to perform a disinfection action efficiently in a plurality of disinfection target areas and complete the disinfection action promptly, an optimum route needs to be analyzed as needed based on the location and time information about the performed disinfection action.

With the system 100 according to the exemplary embodiments, in a case where it is determined that a disinfection action has been performed, the determination result, location information about the disinfection target areas, and time information about the performed disinfection action are stored in association with each other. Consequently, information indicating that the disinfection action has been performed is stored and can be checked, so that omissions of disinfection actions are reduced. Furthermore, which order leads to prompt completion is analyzed based on the location and time of the performed disinfection action, and an optimum order and an optimum route of disinfection by a worker are indicated.

### Determination Unit

The determination unit 101 according to the exemplary embodiments acquires a determination result about whether a disinfection action has been performed. A situation where a disinfection action has been performed according to the exemplary embodiments refers to, for example, a case where a disinfectant is applied to a disinfection target area and the applied disinfectant is wiped or a situation where both the applying and the wiping are performed.

The application of the disinfectant and the wiping of the applied disinfectant may be performed by a person or by a machine.

According to the exemplary embodiments, a determination result about whether a disinfection action has been performed may be acquired using a detection unit described below or by receiving an input from a person having performed a disinfection action or a person having checked that a disinfection action has been performed.

### Storage Unit

The storage unit 102 according to the exemplary embodiments stores a determination result that a disinfection action has been performed in the disinfection target areas, location information about the disinfection target areas, and time information about the performed disinfection action in association with each other. Not only the information indicating that a disinfection action has been performed but also the time and location information about the performed disinfection action are acquired, and this makes it possible to perform disinfection efficiently in the plurality of disinfection target areas.

Examples of the storage unit 102 according to the exemplary embodiments include a storage medium such as a flexible disk, a hard disk, an optical disk, a magneto-optical disk, a compact disk (CD) read-only memory (ROM) (CD-ROM), a CD-recordable (CD-R), a magnetic tape, a non-volatile memory card, or a digital versatile disk (DVD) besides a memory such as a ROM or a random access memory (RAM).

### Time Information About Performed Disinfection Action

The time information about the performed disinfection action according to the exemplary embodiments is not particularly limited and may be any information indicating the time at which the disinfection action has been performed or equivalent information. At least one selected from the group consisting of a time at which the disinfectant has been wiped, a time at which the wiping of the disinfectant has been checked, and an elapsed time since a previous disinfection action is used.

### Location Information About Performed Disinfection Action

The location information about the performed disinfection action according to the exemplary embodiments may be a position at which a disinfection action target item is placed or a room in which a disinfection action target item is placed.

The location information about the performed disinfection action according to the exemplary embodiments is not particularly limited and may be any information that enables identification of the position at which the disinfection action has been performed. Examples of the location information include a target item on which the disinfection action has been performed, a position, an area, and a target item, which are, more specifically, spaces for shared use by an unspecified number of people such as conference rooms, coworking spaces, manga cafes, public facilities, and chairs, tables, and restrooms in the spaces.

### Disinfectant

The disinfectant according to the exemplary embodiments is not particularly limited but is typically a liquid composition that is sprayed to the disinfection target areas. Examples of active ingredients of liquid compositions include surfactants, alkaline components, acidic components, and alcohols. Examples of alkaline components include alkali, sodium hypochlorite, hypochlorous acid, and sodium salts thereof.

### Detection Unit

The system 100 according to the exemplary embodiments may include a detection unit 103 configured to detect environmental change in the disinfection target areas and output information about the environmental change, as illustrated in FIG. 1. In this case, the determination unit 101 can determine whether a disinfection action has been performed in the disinfection target areas based on the information about the environmental change that is output from the detection unit 103.

For example, the information about the environmental change in the disinfection target areas that is output from the detection unit 103 is output as a numerical value, and in a case where the numerical value is greater than the predetermined threshold, it can be determined that a disinfection action has not been performed. In a case where the numerical value is less than the predetermined threshold, it can be determined that a disinfection action has been performed. The threshold may be determined as suitable for the disinfection target areas. The environmental change in the disinfection target areas refers to a change in the disinfection target areas and a change in an environment around the disinfection target areas. The term “around the disinfection target areas” refers to a range that is detectable by the detection unit 103.

The detection unit 103 according to the exemplary embodiments is not particularly limited and may be any detection unit capable of detecting environmental change in the disinfection target areas. At least one unit selected from the group consisting of a unit configured to detect optical change, a unit configured to detect vibration, a unit configured to detect electrical change, and a unit configured to detect temperature change may be used.

### Unit Configured to Detect Optical Change

The unit configured to detect optical change according to the exemplary embodiments detects change in light acquired from the disinfection target areas. Change in light herein refers to at least one of a change in light intensity and a change in light wavelength.

The unit configured to detect optical change according to the exemplary embodiments may be an imaging unit, such as a camera, a video camera, a terminal with a camera function, a smartphone with a camera function, a tablet with a camera function, or a monitoring camera. It is also possible to use a camera (wearable camera, mobile camera) that a worker who performs a disinfection action wears. A typical example of the unit configured to detect optical change is an image information acquisition unit, and either one of an image information acquisition unit that acquires still images and an image information acquisition unit that acquires moving images may be used.

In a case where the image information acquisition unit is configured to acquire moving images, a unit configured to acquire images of the disinfection target areas from moving images acquired during a predetermined period may be included. A unit configured to detect changed points in moving images to acquire images of the disinfection target areas from the moving images may be included. In this case, from the point of view of security, it is desirable to not store images other than the images of the disinfection target areas.

The unit configured to detect optical change may detect optical change in the disinfection target areas without acquiring images. For example, in a case where the disinfectant is liquid, whether the liquid has been applied may be detected using a liquid intrusion indicator. In a case where a disinfectant visualization sheet 403 described below is used, for example, an exhibited color (specific wavelength) of a visualization agent placed in the disinfection target areas may be detected. Alternatively, in a case where the visualization agent is in the form of a two-dimensional code or is placed to indicate text information or image information (diagram) as described below, the two-dimensional code, the text information, or the image information may be detected.

### Unit Configured to Detect Vibration

The unit configured to detect vibration according to the exemplary embodiments may be any unit capable of detecting a vibration caused by a disinfection action, such as a vibration of equipment such as a table or a chair. For example, at least one of an acceleration sensor and a velocity sensor may be used to detect a vibration that indicates that a disinfection action has been performed. Either one of a contact-type acceleration sensor and a non-contact-type acceleration sensor may be used. Contact-type sensors such as piezoelectric sensors, conductive sensors, and strain gauge sensors may be used. Non-contact-type sensors such as eddy current sensors and displacement detection sensors such as capacitive sensors and optical sensors may be used.

### Unit Configured to Detect Electrical Change

The unit configured to detect electrical change according to the exemplary embodiments detects electrical change in current or voltage caused by application of the disinfectant. Examples thereof include pH meters, alcohol sensors, and circuits with current and voltage that are changed by application of a liquid.

### Unit Configured to Detect Temperature Change

The unit configured to detect temperature change according to the exemplary embodiments detects temperature change in the disinfection target areas or areas near the disinfection target areas. For example, a temperature decrease caused by vaporization heat after application of the disinfectant such as alcohol to the disinfection target areas may be detected, or a temperature increase in a disinfection target item while a person is wiping the disinfectant applied to the disinfection target item may be detected. A publicly-known temperature sensor may be used as the unit configured to detect temperature change according to the exemplary embodiments.

In the system 100 according to the exemplary embodiments, the determination unit 101 and the storage unit 102 may be configured to communicate data (information) with each other via communication units (104, 105) as illustrated in FIG. 1. With this configuration, the determination result that a disinfection action has been performed in the disinfection target areas and the time and location information about the performed disinfection action are transmitted by the terminal communication unit 104 of a terminal apparatus 110. The transmitted data is received by the server communication unit 105 of a server apparatus 120. The received data is stored in the storage unit 102.

### Analysis Unit

An analysis unit 106 performs analysis based on the determination result, the location information about the performed disinfection action, and the time information about the performed disinfection action that are stored in the storage unit 102. For example, the analysis unit 106 is capable of outputting recommendation information about a recommended disinfection order in performing disinfection actions in the plurality of disinfection target areas. Examples of recommendation information include an order that leads to the fastest disinfection action completion.

### Terminal Communication Unit, Server Communication Unit

The terminal communication unit 104 and the server communication unit 105 according to the exemplary embodiments communicate information (data) such as determination results. For example, a local area network (LAN) may be used, which may be either one of a wired LAN or a wireless LAN, or a wide area network (WAN) may be used. The terminal communication unit 104 is realized by, for example, a wireless communication module and controls communication with the server communication unit 105 and communication networks. The same applies to the server communication unit 105.

### Terminal Apparatus

An example of the terminal apparatus 110 according to the exemplary embodiments is an apparatus including the detection unit 103, the determination unit 101, and the terminal communication unit 104 (FIG. 1). The terminal apparatus 110 according to the exemplary embodiments transmits at least a determination result that a disinfection action has been performed to the server apparatus 120 in a case where a disinfection action has been performed.

The terminal apparatus 110 according to the exemplary embodiments may be a dedicated terminal for performing the disinfection action determination or a general-purpose smartphone or tablet.

### Server Apparatus

The server apparatus 120 according to the exemplary embodiments communicates information (data) with the terminal apparatus 110 in the disinfection target areas and has functions such as storage and analysis functions.

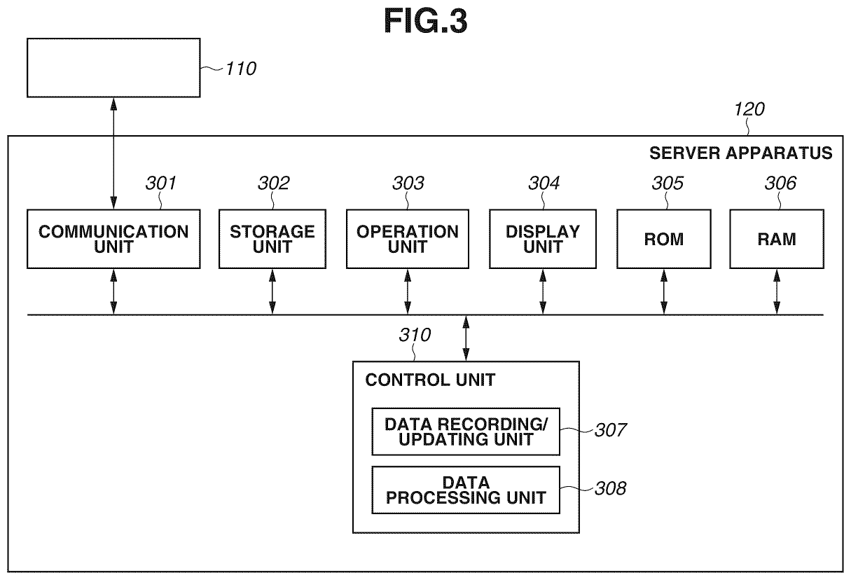

An example of the server apparatus 120 will be described below with reference to a schematic diagram in FIG. 3. The server apparatus 120 includes a communication unit 301 (server communication unit 105), a storage unit 302 (storage unit 102), an operation unit 303, a display unit 304, a ROM 305, a RAM 306, and a control unit 310. The communication unit 301 is realized by a LAN card and controls communication with external apparatuses (e.g., terminal apparatus 110) and communication networks. The ROM 305 is realized by a non-volatile memory and stores various programs. The RAM 306 is realized by a volatile memory and temporarily stores various information. The storage unit 302 is realized by a hard disk drive (HDD), a solid state drive (SSD), or a network attached storage (NAS) and stores various information. The operation unit 303 is realized by a keyboard and/or a mouse, and the display unit 304 is realized by a display and displays various information to users.

The control unit 310 includes a data recording/updating unit 307 and a data processing unit 308. The data recording/updating unit 307 records the determination result that a disinfection action has been performed, time information about the performed disinfection action, and location information about the performed disinfection action in the storage unit 302, the ROM 305, and/or the RAM 306. The data processing unit 308 refers to, for example, databases stored in the storage unit 302, the ROM 305, and/or the RAM 306, adds information to read information, converts read information, and/or performs data processing on read information. The processing results may be communicated to the terminal apparatus 110 using the communication unit 301.

### Method

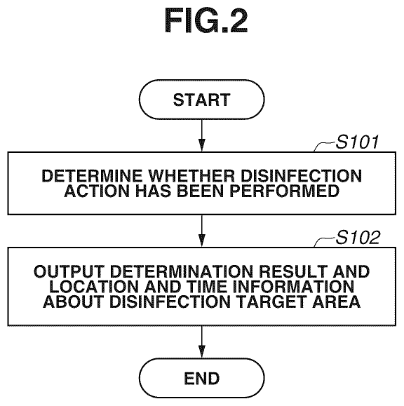

FIG. 2 is a flowchart illustrating a method for assisting in a disinfection action in the plurality of disinfection target areas according to the exemplary embodiments and including at least the following processes.
(1) A process of determining whether a disinfection action has been performed in the disinfection target areas. and outputting the determination result (step S101).
(2) A process of storing, in a case where it is determined that a disinfection action has been performed in the disinfection target areas, the determination result output in the process (1), location information about the disinfection target areas, and time information about the performed disinfection action in association with each other (step S102).

### First Exemplary Embodiment

The system 100 according to a first exemplary embodiment will be described below with reference to FIG. 1. A case where the detection unit 103 according to the present exemplary embodiment is a monitoring camera capable of detecting environmental change in the disinfection target areas will be described below as an example.

According to the present exemplary embodiment, a camera 103 is used as the detection unit 103 to detect environmental change, i.e., a disinfection action has been performed, in the disinfection target areas. In this case, it is determined that a disinfection action has been performed in a case where an image characteristic of a disinfection action is acquired. For example, in a case where it is determined that a worker has performed wiping of the disinfectant, it is determined that a disinfection action has been performed. The determination result that a disinfection action has been performed is transmitted to the server apparatus 120 together with time information about the time determined as the time at which the disinfection action has been performed, and location information about the performed disinfection action. The storage unit 102 of the server apparatus 120 stores the determination result, the time information about the time determined as the time at which the disinfection action has been performed, and the location information about the performed disinfection action in association with each other.

The storing in the storage unit 102 is performed similarly for the plurality of disinfection target areas.

The analysis unit 106 provides information (recommendation information) about a recommended order of performing disinfection for the plurality of disinfection target areas based on the information stored in the storage unit 102.

According to the present exemplary embodiment, the monitoring camera is used, so that no new devices for collecting information for the disinfection action determination are necessary.

### Second Exemplary Embodiment

A system according to a second exemplary embodiment uses the disinfectant visualization sheet 403 in the determination of whether a disinfection action has been performed, in the system according to the first exemplary embodiment. Application of the disinfectant to the disinfectant visualization sheet 403 according to the present exemplary embodiment visualizes the disinfectant visualization sheet 403. The disinfectant visualization sheet 403 will be described specifically below.

### Disinfectant Visualization Sheet

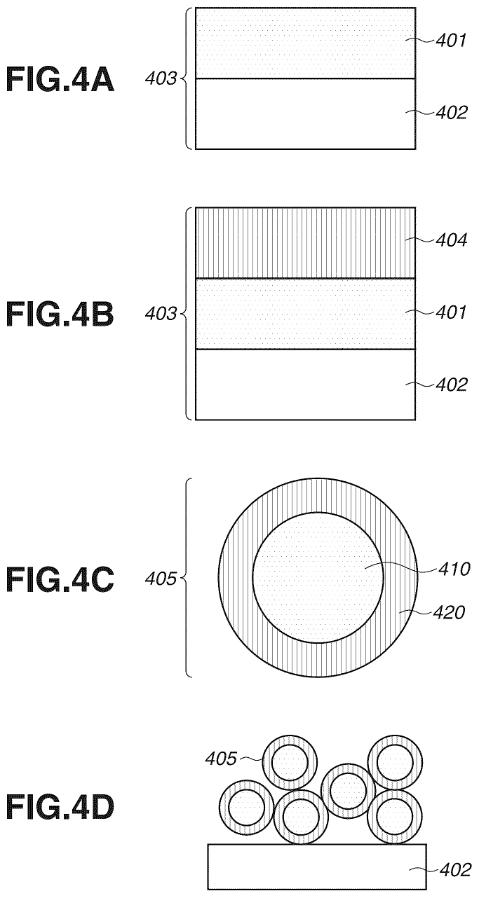

The disinfectant visualization sheet 403 according to the present exemplary embodiment includes a substrate 402 and a color reaction portion 401 disposed on the substrate 402 as illustrated in FIG. 4A. The color reaction portion 401 includes a visualization agent (not illustrated), and application of the disinfectant to the visualization agent changes a color reaction state of the visualization agent. Use of the disinfectant visualization sheet 403 according to the present exemplary embodiment as described above makes it possible to check whether the disinfectant has been applied, by visually checking a change in the color reaction state, which is convenient. The situation in which the disinfectant and the visualization agent are mixed together is when the disinfectant is applied to the disinfectant visualization sheet 403, and the time during which the disinfectant and the visualization agent are mixed together is short. This improves the long-lasting preservation of materials that are used.

### Color Reaction Portion

The color reaction portion 401 according to the present exemplary embodiment includes at least the visualization agent. The color reaction portion 401 may include a binder to enhance adhesion with the substrate 402. A resin may be used as the binder. For example, urethane resin or polyvinyl alcohol may be used.

The color reaction portion 401 desirably has a thickness of 1 μm or more and 80 μm or less, more desirably 5 μm or more and 60 μm or less, particularly desirably 15 μm or more and 50 μm or less. The color reaction portion 401 that is thicker than 80 μm is undesirable because adhesion with the substrate 402 decreases. The color reaction portion 401 that is thinner than 1 μm is undesirable because sufficient color reaction cannot be achieved.

### Visualization Agent

The visualization agent according to the present exemplary embodiment is not particularly limited and may be any visualization agent that reacts with the disinfectant to cause a change in the color reaction state. A change in the color reaction state according to the present exemplary embodiment refers to a change in color that is visually detectable before and after application of the disinfectant. Examples of the change include an occurrence of at least one of a change from a visually undetectable state to a visually detectable state, a change from a visually detectable state to a visually undetectable state, and a change in color that is visually detectable. A change in color that is visually detectable herein includes a change in color shade.

A phenomenon of change in the color reaction state is chromism. Chromism includes photochromism, thermochromism, electrochromism, acidochromism, solvatochromism, and vaporochromism. According to the present exemplary embodiment, substances that cause the phenomena are referred to as chromic substances, particularly as photochromic substances, thermochromic substances, electrochromic substances, acidochromic substances, solvatochromic substances, and vaporochromic substances.

Acidochromic substances change color due to a pH change, and the color range of each substance varies depending on properties of the substance. An example of the substance is at least one type selected from the group consisting of Metanil Yellow, Metacresol Purple, Thymol Blue, Tropaeolin O, 2,4-dinitrophenol, Methyl Yellow, Bromophenol Blue, Congo Red, Methyl Orange, Bromochlorophenol Blue, Alizarin Red S, Bromocresol Green, Methyl Orange-Xylene Cyanol FF, 2,5-dinitrophenol, Methyl Orange-Indigo Carmine, Methyl Red, Methyl Orange-Xylene Cyanol FF-phenolphthalein, lacmoid, Chlorophenol Red, o-nitrophenol, p-nitrophenol, Bromocresol Green-Methyl Red, Bromocresol Purple, Bromophenol Red, Methyl Red-Methylene Blue, Bromothymol Blue, Neutral Red, Phenol Red, Neutral Red-Bromothymol Blue, Cresol Red, alpha-naphtholphthalein, Bromothymol Blue-Phenol Red, curcumin, phenolphthalein, Cresol Red-Thymol Blue, o-Cresolphthalein, alpha-Naphtholbenzein, Thymolphthalein, Thymol Blue-phenolphthalein, Alizarin Yellow GG, Alizarin Yellow R, Tropaeolin O, nitramine, 1,3,5-trinitrobenzene, Indigo Carmine, Methyl Violet, litmus, and Methyl Purple. There are substances that change color in one of acidic and alkaline pH ranges, while there are other substances that change color in both acidic and alkaline pH ranges, such as Thymol Blue. Substances that have a low level of toxicity and are more desirable are Metanil Yellow, Metacresol Purple, Thymol Blue, Tropaeolin O, Bromophenol Blue, Bromochlorophenol Blue, Alizarin Red S, Bromocresol Green, Methyl Red, lacmoid, Chlorophenol Red, o-Nitrophenol, Bromocresol Purple, Bromophenol Red, Bromothymol Blue, Neutral Red, Phenol Red, Cresol Red, alpha-Naphtholphthalein, phenolphthalein, o-Cresolphthalein, thymolphthalein, Alizarin Yellow GG, Alizarin Yellow R, Tropaeolin O, Methyl Violet, litmus, and Methyl Purple. Substances that are more desirable due to the reason of change color at a pH from 3 to 11 inclusive are Metacresol Purple, Thymol Blue, Tropaeolin O, Bromophenol Blue, Bromochlorophenol Blue, Alizarin Red S, Bromocresol Green, Methyl Red, lacmoid, Chlorophenol Red, o-Nitrophenol, Bromocresol Purple, Bromophenol Red, Bromothymol Blue, Neutral Red, Phenol Red, Cresol Red, alpha-Naphtholphthalein, phenolphthalein, o-Cresolphthalein, thymolphthalein, Alizarin Yellow GG, Alizarin Yellow R, litmus, and Methyl Purple. Desirably, alkaline disinfectants change color in alkaline conditions, and acidic disinfectants change color in acidic conditions.

Further, as the disinfectant evaporates, the visualization agent changes color and is restored. The restoration herein refers to the returning to a former state before the application of the disinfectant. In other words, the color reaction state is reversible. Specifically, the visualization agent according to the present exemplary embodiment that changes its color reaction state for a predetermined time upon application of the disinfectant and thereafter returns to the former state before the application of the disinfectant may be used. For example, the color reaction state of the visualization agent in a visually undetectable state changes to a visually detectable state upon application of the disinfectant, and after the predetermined time passes, the color reaction state returns to the visually undetectable state. Being reversible as described above, the visualization agent may be used in situations where disinfection needs to be performed repeatedly.

Two or more types of visualization agents may be used. For example, the use of visualization agents that maintain their displayed colors for different durations from each other is desirable because this makes it possible to display a time from the contact with the disinfectant as an indicator.

The visualization agent may be a material outside the visible light range, and a material that absorbs light with ultraviolet and infrared wavelengths may be used. The ultraviolet and infrared ranges include near-ultraviolet and near-infrared ranges. In this case, a system relevant to the observation is provided to the monitoring camera. The material and the observation system that are publicly known are applicable.

### Substrate

The substrate 402 according to the present exemplary embodiment may be any substrate on which the color reaction portion 401 is able to be disposed. The substrate 402 that is a resin sheet is suitable because it is lightweight and flexible. Examples of resin sheets include polyester resins such as polyethylene terephthalate, polybutylene terephthalate, and polyethylene terephthalate/isophthalate copolymers, polyolefin resins such as polyethylene, polypropylene, and polymethylpentene, polyethylene fluoride resins such as polyvinyl fluoride, polyvinylidene fluoride, polytetrafluoroethylene, and ethylene-tetrafluoroethylene copolymers, aliphatic polyamide resins such as Nylon 6 and Nylon 6,6, vinyl polymer resins such as polyvinyl chloride, vinyl chloride/vinyl acetate copolymers, ethylene/vinyl acetate copolymers, ethylene/vinyl alcohol copolymers, polyvinyl alcohol, and vinylon, cellulose-based resins such as triacetate cellulose and cellophane, acrylic resins such as polymethyl methacrylate, polyethyl methacrylate, polyethyl acrylate, and polybutyl acrylate, and other synthetic resins such as polystyrene, polycarbonate, polyarylate, and polyimide. One type of a resin sheet may be used alone, or two or more types of resin sheets may be combined or layered and used. Desirably, the substrate 402 is provided with a reception layer for retaining the color reaction portion 401 because in a case where the visualization agent is ink, it is easy for the reception layer to retain the visualization agent.

Desirably, the reception layer of the substrate 402 according to the present exemplary embodiment does not react with the visualization agent. For example, in a case where the visualization agent displays color in alkaline conditions, the reception layer is desirably neutral or acidic. The reception layer that reacts with the visualization agent is undesirable because which causes the visualization agent to exhibit a color reaction prior to a reaction with the disinfectant.

The substrate 402 according to the present exemplary embodiment may be transparent or opaque or may have a color. In a case where the visualization agent has a color before reacting with the disinfectant, the substrate 402 desirably has a color close to the color of the visualization agent.

Release paper, metal sheet, or wood may be used as the substrate 402 according to the present exemplary embodiment. An adhesive portion may be provided to bond the substrate 402 to another member.

### Retention Portion

The disinfectant visualization sheet 403 according to the present exemplary embodiment may include a retention portion 404 on the color reaction portion 401 to retain the disinfectant as illustrated in FIG. 4B. The retention portion 404 makes it possible to extend the time during which the disinfectant is retained, and this makes it possible to extend the time during which the disinfectant reacts with the visualization agent and color is exhibited. Further, the color reaction time is controllable by suitably selecting materials to be provided to the retention portion 404. For example, the time of changing from the visually detectable color reaction state to the visually undetectable state is controlled.

Having the foregoing function, the retention portion 404 may also be referred to as fading time control portion.

Desirably, the retention portion 404 includes empty spaces to retain the applied disinfectant. For example, the retention portion 404 may be formed using a resin and inorganic particles that form empty spaces. The inorganic particles for use herein are of at least one type selected from the group consisting of pearl necklace-shaped silica particles, chain-like silica particles, spherical colloidal silica, non-spherical colloidal silica, alumina particles, titania particles, and zirconia particles. Desirably, a peak position of a particle size distribution of particle diameters of inorganic fine particles analyzed by dynamic light scattering is at 1 nm or more and 300 nm or less, more desirably 1 nm or more and 150 nm or less, particularly desirably 1 nm or more and 60 nm or less. Especially for pearl necklace-shaped silica particles, the peak position is desirably at 20 nm or more and 50 nm or less. As for chain-like silica particles, the peak position is desirably at 1 nm or more and 10 nm or less. Large particle diameters are undesirable because empty spaces decrease. Particle diameters that are excessively small are also undesirable because pore diameters of empty spaces formed by inorganic particles become so small that the applied disinfectant is absorbed at low speed. Desirably, a pore diameter distribution curve of the pore diameters has a maximum peak in the range of 5 nm or more and 20 nm or less inclusive. With a peak of pore diameters at 5 nm or less, the absorption rate of the disinfectant becomes slow, whereas with a peak beyond 20 nm, haze may increase. In a case where haze increases, whitishness becomes noticeable, and the visibility of the color reaction portion 401 decreases. Thus, this is undesirable. Pore diameters can be measured by BET specific surface area measurement.

While resins for use herein may be selected from various resins, in a case where the disinfectant is a water-based disinfectant, a water-soluble resin is desirable. For example, at least one type of a water-soluble resin selected from the group consisting of cellulose-based binders such as methylcellulose, methylhydroxyethyl cellulose, methylhydroxypropyl cellulose, and hydroxyethyl cellulose, starch and modified products thereof, gelatin and modified products thereof, natural polymer resins such as casein, pullulan, Arabic gum, karaya gum, and albumin or derivatives thereof, polyvinyl alcohol and modified products thereof, latex and emulsions such as styrene-butadiene rubber (SBR) latex, nitrile butadiene rubber (NBR) latex, methyl methacrylate-butadiene copolymers, and ethylene-vinyl acetate copolymers, polyacrylamide, vinyl polymers such as polyvinylpyrrolidone, polyethyleneimine, polypropylene glycol, polyethylene glycol, and maleic anhydride or copolymers thereof may be used.

The thickness of the retention portion 404 does not need to be uniform. For example, the disinfectant retention time is controllable by changing the thickness of a portion. This is desirable because such a configuration makes it possible to control the color reaction time of the color reaction portion 401, and the time from the contact with the disinfectant can be displayed as an indicator.

### Protection Portion

According to the present exemplary embodiment, a protection portion for protecting the color reaction portion 401 may be provided on the color reaction portion 401. The phrase “on the color reaction portion 401” herein indicates that the protection portion may be provided in contact with the color reaction portion 401 or, in a case where the retention portion 404 is provided on the color reaction portion 401, the protection portion may be provided on the retention portion 404. For example, in a case where a water-soluble resin is used as the retention portion 404, since a surface becomes viscous, it is desirable to coat with the protection layer. At least one type selected from the group consisting of resins such as acrylic resins, vinyl acetate resins, vinyl chloride resins, ethylene/vinyl acetate copolymer resins, polyamide resins, polyester resins, urethane resins, polyolefin resins, and copolymer resins thereof may be used as the protection portion according to the present exemplary embodiment.

Since the surface of the disinfectant visualization sheet 403 is wiped with the disinfectant repeatedly, the fading time control portion is desirable because it provides durability.

### Two-Dimensional Code

The disinfectant visualization sheet 403 according to the present exemplary embodiment may include the visualization agent in the shape of a two-dimensional code.

The two-dimensional code is at least one two-dimensional code selected from the group consisting of Quick Response code (QR code®), micro QR code, SP code, VeriCode, MaxiCode, CP code, DataMatrix, DataMatrix ECC200, Code1, AztecCode, INTACTA.CODE, card e, Chameleon Code, PDF417, micro PDF417, Code49, Code16K, Codablock, SuperCode, UltraCode, RSS Composite, and AztecMesa. With the visualization agent being provided to form a two-dimensional code on the substrate 402, the imaging unit reads the two-dimensional code, and the determination of whether the disinfectant has been applied is performed based on the image information.

From the two-dimensional code, information about the time of application of the disinfectant and information about the location of application of the disinfectant are obtainable. Upon reading the two-dimensional code, text information or image information indicating completion of disinfection (cleaning) may be displayed on a display unit of a terminal having read the two-dimensional code. In a case where a disinfection target area is a place that requires payment of a fee, such as a store or a coworking space, reading the two-dimensional code may connect to an attached fee payment system. Upon reading the two-dimensional code, information such as text information indicating that points that can be used at the store are provided or returned, that a discount is granted, or a gift is presented may be displayed to motivate the user to perform disinfection (cleaning).

### Another Example of Disinfectant Visualization Sheet

In another example of the disinfectant visualization sheet 403 according to the present exemplary embodiment, the color reaction portion 401 may be provided with visualization particles including the visualization agent and carrier particles carrying the visualization agent. An agglomerate of visualization particles is configured to function as the color reaction portion 401. In a case where the visualization particles are used, positions of a hydration layer and a layer including the visualization particles can be designed microscopically, which enables precise characteristic design.

### Invisible Portion

In yet another example of the disinfectant visualization sheet 403 according to the present exemplary embodiment, a combination of a plurality of principles may be used in addition to the visualization agent. A sheet including the above-described visualization color reaction portion may be provided with an invisible portion. This is a form in which, for example, materials of the visualization agent that are outside the visible light range are applied in combination. The invisible portion according to the present exemplary embodiment may be provided with invisible materials that absorb light with ultraviolet and infrared wavelengths. The ultraviolet and infrared ranges include near-ultraviolet and near-infrared ranges. With the invisible agent, additional checking from a third-party perspective becomes possible in addition to visual checking of whether the disinfectant has been applied.

Furthermore, the disinfectant visualization sheet 403 according to the present exemplary embodiment may be used in further combination with thermochromism. For example, the visualization color reaction portion is used in combination with a dye that changes color in response to temperature. Consequently, in a case where there is a change in temperature from a use environment, the exhibited color further changes in addition to the color change based on the presence or absence of the disinfectant. This leads to recognition of a touch history indirectly, for example. A thermochromism dye may be selected from publicly-known substances.

As a dye that absorbs the infrared spectrum among the invisible materials according to the present exemplary embodiment, at least one type selected from the group consisting of phthalocyanine dyes, naphthalocyanine dyes, metal complex dyes, polymethine dyes, quinone dyes, azo dyes, diphenylmethane and triphenylmethane dyes, radical dyes, perimidine dyes, and Au nanorods may be used. As a dye that absorbs the ultraviolet spectrum and emits fluorescence, at least one type selected from the group consisting of melocyanin, perylene, acridine, luciferin, pyranine, stilbene, rhodamine, coumarin, and fluorescein may be used.

### Visually Detectable Wavelength Range

A visually detectable wavelength range according to the present exemplary embodiment refers to light in the wavelength range from 360 nm or more to 830 nm or less inclusive. The infrared wavelength range is from 900 nm or more to 14 μm or less inclusive, and the ultraviolet wavelength range is from 100 nm or more to 400 nm or less inclusive.

### Marking Composition

The color reaction portion 401 of the disinfectant visualization sheet 403 according to the present exemplary embodiment may be provided as a marking indicating whether the disinfectant has been applied (cleaned). A material for marking according to the present exemplary embodiment may be referred to as marking composition. The marking composition contains the visualization agent and a solvent, and the solvent may be a hydrophobic solvent or an aqueous solvent. The hydrophobic solvent may be an organic solvent such as heptane or petroleum ether, and the aqueous solvent may be water or alcohol.

As the aqueous solvent according to the present exemplary embodiment, water or a mixed medium using water as a major solvent in combination with a protonic organic solvent or a non-protonic organic solvent may be used. It is desirable to use a medium that mixes with or dissolves in water at any proportion, desirably a uniform mixed medium containing 50% or more by mass of water, as the organic solvent according to the present exemplary embodiment. As the water, deionized water (ion-exchanged water) or ultrapure water is desirably used.

The protonic organic solvent is an organic solvent containing hydrogen atoms (acidic hydrogen atoms) bonded to oxygen or nitrogen. The non-protonic organic solvent is an organic solvent without an acidic hydrogen atom. Examples of organic solvents include alcohols, alkylene glycols, polyalkylene glycols, glycol ethers, glycol ether esters, carboxylic acid amides, ketones, keto alcohols, or cyclic ethers.

Examples of aqueous mediums that are suitable for use include water, water/ethanol mixed solvents, water/ethylene glycol mixed solvents, or water/N-methylpyrrolidone mixed solvents. The water content based on the total mass of the composition is desirably 10.0% or more by mass and 90.0% or less by mass, desirably 50.0% or more by mass and 90.0% or less by mass.

The content of the water-soluble organic solvent in the composition based on the total mass of the composition is desirably 5.0% or more by mass and 90.0% or less by mass, more desirably 10.0% or more by mass and 50.0% or less by mass.

### Other Additives

The marking composition according to the present exemplary embodiment may further contain, in addition to the above-described components as needed, a water-soluble organic compound such as polyhydric alcohols such as trimethylolpropane and trimethylolethane, urea, or urea derivatives such as urea and ethylene urea. Furthermore, the marking composition according to the present exemplary embodiment may contain various additives as needed such as a surfactant, a pH adjuster, a rust inhibitor, an anticorrosive agent, an antimicrobial agent, an antioxidant, a reduction inhibitor, an evaporation enhancer, a chelating agent, and a water-soluble resin. The surfactant may be an anionic, cationic, or nonionic surfactant. The content of the surfactant in the marking composition based on the total mass of the composition is desirably 0.1% or more by mass and 5.0% or less by mass, more desirably 0.1% or more by mass and 2.0% or less by mass. Specific examples of surfactants that are suitable for use include nonionic surfactants such as polyoxyethylene alkyl ether, polyoxyethylene fatty acid ester, polyoxyethylene alkyl phenyl ether, polyoxyethylene-polyoxypropylene block copolymers, and acetyleneglycol-based compounds.

In recording images on recording mediums by discharging the marking composition according to the present exemplary embodiment from an inkjet recording head, it is desirable to use the marking composition that has an appropriately-controlled surface tension and an appropriately-controlled viscosity. Specifically, the density of colorant compounds in the marking composition is desirably about 5% to about 20%. The surface tension of the composition at 25° C. is desirably 10 mN/m or more and 60 mN/m or less, more desirably 20 mN/m or more and 60 mN/m or less, particularly desirably 30 mN/m or more and 50 mN/m or less. The viscosity of the composition at 25° C. is desirably 1.0 mPa·s or more and 10 mPa·s, more desirably 1.0 mPa·s or more and 5 mPa·s or less.

### Image Recording Method [Marking Composition Application Method]

An image recording method according to the present exemplary embodiment relates to a method for applying the marking composition to a substrate. Various methods may be used in the image recording method according to the present exemplary embodiment, and examples thereof include inkjet methods, flexographic method, screen methods, offset methods, and spin coating methods. For example, inkjet methods are methods for recording images on recording mediums by discharging the marking composition according to the present exemplary embodiment from an inkjet recording head. A method for discharging the marking composition may be a method in which mechanical energy is applied to the marking composition and a method in which thermal energy is applied to the composition. Publicly-known methods may be used in the processes of the inkjet recording method, except that the marking composition according to the present exemplary embodiment is used.

### Disinfectant Visualization Particles

A color reaction state of disinfectant visualization particles 405 according to the present exemplary embodiment changes upon application of the disinfectant. Specifically, the disinfectant visualization particles 405 each include a visualization agent 420 and a carrier particle 410 carrying the visualization agent 420 as illustrated in FIG. 4C. The color reaction state of the visualization agent 420 changes upon application of the disinfectant. The disinfectant visualization particles 405 according to the present exemplary embodiment may include the fading time control portion (not illustrated). The fading time control portion does not necessarily need to be layered. Porous particles may be used as the fading time control portion. For example, at least one type selected from the group consisting of mesoporous silica particles, porous silica particles, porous titania particles, porous zirconia particles, porous ceria particles, porous zinc oxide particles, porous crosslinked polymethyl methacrylate particles, porous crosslinked polystyrene particles, and porous methyl methacrylate-styrene copolymer crosslinked particles may be used. By impregnating the porous particles with a solution in which the visualization agent is dissolved and then drying the porous particles, the disinfectant visualization particles 405 including the color reaction portion 401 are formed.

The porous particles may be treated to have a hydrophilic or hydrophobic surface. In a case where the disinfectant is a water-based disinfectant, hydrophilic surfaces are desirable, whereas in a case where the disinfectant is not a water-based disinfectant, hydrophobic surfaces are desirable.

Since the disinfectant visualization particles 405 have high rigidity, the durability remains high even after wiping of the disinfectant is performed repeatedly, so that using the disinfectant visualization particles 405 is desirable.

The particle diameter of the disinfectant visualization particles 405 is desirably 30 nm or more and 5 μm or less, more desirably 50 nm or more and 3 μm or less, particularly desirably 80 nm or more and 1 μm or less. A particle diameter less than 30 nm is undesirable because dispersion efficiency is low, which raises an issue in long lasting stability. A particle diameter greater than 5 μm is undesirable because sedimentation often occurs in dispersing the disinfectant visualization particles 405 in a solution.

### Sheet Using Disinfectant Visualization Particles

The disinfectant visualization particles 405 may be provided on the substrate 402 as illustrated in FIG. 4D to use the disinfectant visualization particles 405 according to the present exemplary embodiment as a sheet using the disinfectant visualization particles 405.

### Coating Liquid and Coated Article With Disinfectant Visualization Particles

The disinfectant visualization particles 405 according to the present exemplary embodiment may be used as a coating liquid such as a dispersion liquid or a slurry. The coating liquid may be applied to an article to form a coated article.

### Modified Example of Disinfectant Visualization Particles

The disinfectant visualization particles 405 according to the present exemplary embodiment do not necessarily need to be in the shape of particles. For example, a rod shape, a plate shape, or a film shape may be employed. In the case of a film shape, it is especially desirable to provide the fading time control portion in order to maintain strength against wiping of the disinfectant.

### Third Exemplary Embodiment

A report on a disinfection action according to a third exemplary embodiment will be described below. A screen that is displayed on a display will be described below as an example.

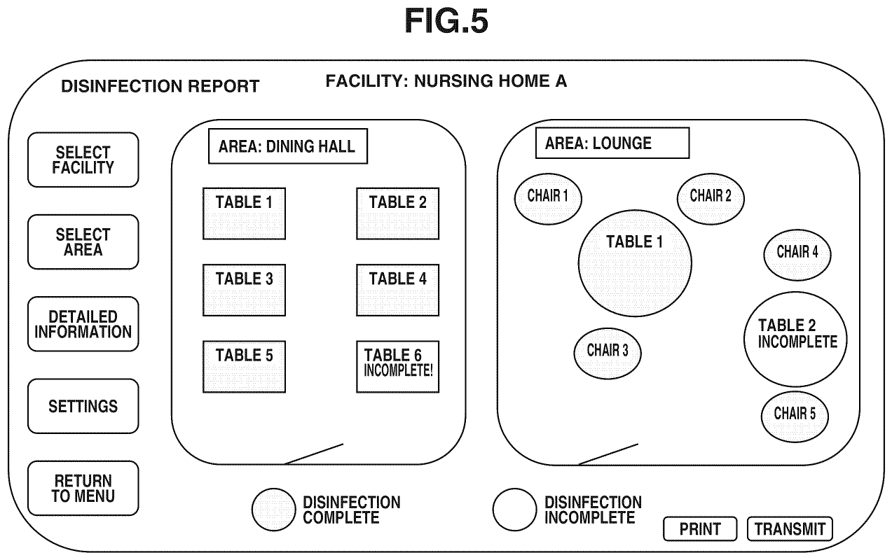

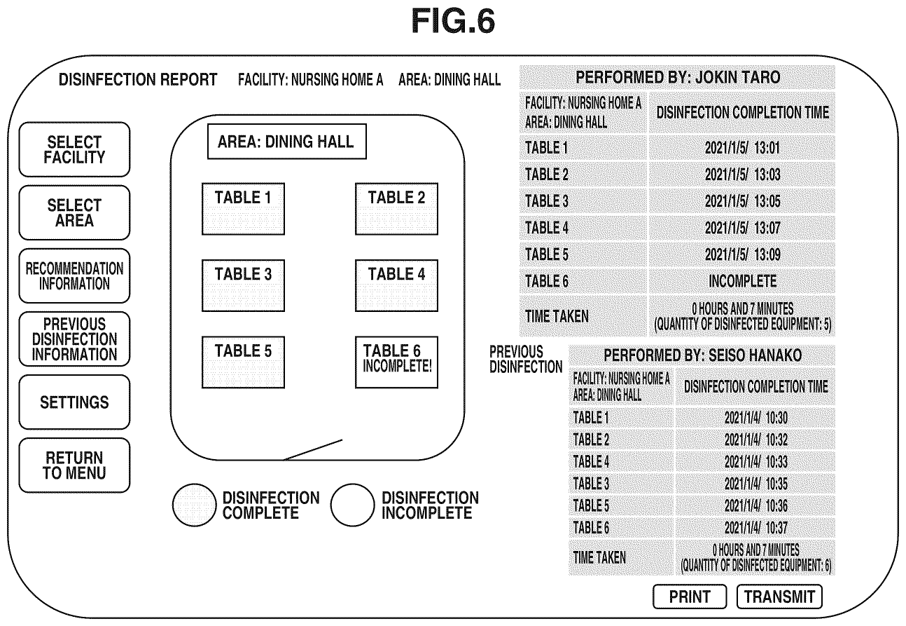

The report on a disinfection action according to the present exemplary embodiment indicates whether a disinfection action has been performed (whether the disinfection action is completed), time information about the performed disinfection action, and location information about the performed disinfection action. FIGS. 5 and 6 illustrate an example of the report. In FIG. 5, a map indicating whether a disinfection action has been performed in a facility or area selected by the user is displayed. Display forms are not particularly limited, and any display forms that clearly indicate equipment with complete disinfection (tables, chairs) and equipment with incomplete disinfection may be used. Not only the illustrated display form based on the presence or absence of hatching but also a display form based on differences in color may be employed. Equipment with incomplete disinfection may be highlighted and displayed as in FIGS. 5 and 6. While this example presents information about whether a disinfection action on equipment is complete or incomplete, information indicating completeness or incompleteness may be displayed for each area.

While the screen that is displayed on the display is described in the example, disinfection action information may be printed on paper and output, or the information displayed on the screen may be transmitted via email.

FIG. 6 illustrates an example of a report that is displayed in a case where detailed information is selected in the disinfection report illustrated in FIG. 5. In this example, a display of detailed information about a dining hall area of nursing home A is selected. Names of persons responsible for disinfection actions in the area and chronological disinfection completion time information about a plurality of disinfection target areas are displayed. The time taken from the first disinfection action completion time to the last disinfection action completion time is displayed, and the quantity of disinfected equipment is also displayed.

As described above, the chronological disinfection completion time information about the disinfection target areas and the time taken are displayed, which makes it possible to identify which order of disinfection actions leads to fast completion of the disinfection actions. In a case where there is a large amount of such data, the analysis unit 106 may specify an optimum order using machine learning.

As described above, the system according to the exemplary embodiments of the present invention provides assistance to perform disinfection efficiently and thoroughly without omissions in actual situations.

### Modified Examples

### Issues in Modified Examples

As a measure against infectious diseases, the importance of disinfection is increasing. Japanese Patent Application Laid-Open No. 2017-169613 discusses a mobile autonomous robot that detects a disinfection target substance with a sensor and automatically sprays hypochlorous acid for disinfection and deodorization. The robot is capable of determining a target item, location, unit, quantity, and time of the supply of hypochlorous acid based on information detected by a chemical sensor and information acquired from an image recognition unit. However, the robot discussed in Japanese Patent Application Laid-Open No. 2017-169613 does not check whether a disinfection action has been performed, i.e., hypochlorous acid has been sprayed to a target item and the sprayed hypochlorous acid has been wiped. In a case where stains are visually detectable, it is easy to check whether a disinfection action has been performed. However, in a case where a disinfection target item is without such stains, it is difficult to check whether a disinfection action has been performed. A person having performed disinfection may perform checking and recording, but it is inconvenient to perform the plurality of processes of checking the disinfection action and recording the disinfection action. Thus, a modified example of the invention is directed to providing a system that makes it easy to check whether a disinfection action has been performed.

### System According to Modified Example

The system 100 according to the present exemplary embodiments includes at least the detection unit 103 and the determination unit 101 as illustrated in FIG. 1. The detection unit 103 detects environmental change in disinfection target areas and output information about the environmental change, and the determination unit 101 determines whether a disinfection action has been performed in the disinfection target areas based on the information about the environmental change. For example, the information about the environmental change in the disinfection target areas that is output from the detection unit 103 is output as a numerical value, and in a case where the numerical value is greater than the predetermined threshold, it can be determined that a disinfection action has not been performed. In a case where the numerical value is less than the predetermined threshold, it can be determined that a disinfection action has been performed. The threshold may be determined as suitable for the disinfection target areas. The environmental change in the disinfection target areas refers to a change in the disinfection target areas and a change in an environment around the disinfection target areas. The term “around the disinfection target areas” refers to a range that is detectable by the detection unit 103.

In the system 100 according to the present exemplary embodiments, the detection unit 103 detects environmental change in the disinfection target areas, and the determination unit 101 determines whether a disinfection action has been performed based on information about the detected environmental change. Thus, it is unnecessary to perform the plurality of processes of checking a disinfection action and recording the disinfection action, whereby whether a disinfection action has been performed is easily checked.

In a case where a person having performed disinfection is to check whether a disinfection action has been performed, the greater the number of disinfection target areas, the higher the likelihood of mistakes in the recording. With the system according to the present exemplary embodiments, on the other hand, the person having performed disinfection does not need to perform the checking, so that the likelihood of mistakes in the recording decreases.

The system 100 according to the present exemplary embodiments may include the storage unit 102. In a case where it is determined that a disinfection action has been performed in the disinfection target areas, the storage unit 102 stores the determination result output from the determination unit 101, location information about the disinfection target areas, and time information about the occurrence of the disinfection action in association with each other. With the information being stored in the storage unit 102, whether a disinfection action has been performed can be checked without relying on human memory, so that in a case where there is an omission of a disinfection action, the omission is detected.

In order to realize an efficient disinfection action, it is desirable to store the location and time information about the performed disinfection action. In an environment with a plurality of disinfection target areas, in order to perform disinfection efficiently, a suitable order of disinfection actions needs to be selected. In a case where there is no change in a disinfection action target environment, disinfection may be performed in a predetermined order. However, in actual situations, there may be an initially unexpected obstacle, or a layout in the environment may be changed periodically. In such actual situations, in order to perform a disinfection action efficiently in a plurality of disinfection target areas and complete the disinfection action promptly, an optimum route needs to be analyzed as needed based on the location and time information about the performed disinfection action.

With the system 100 according to the present exemplary embodiments, in a case where it is determined that a disinfection action has been performed, the determination result, location information about the disinfection target areas, and time information about the performed disinfection action are stored in association with each other. Consequently, information indicating that the disinfection action has been performed is stored and can be checked, so that omissions of disinfection actions are reduced. Furthermore, which order leads to prompt completion is analyzed based on the location and time of the performed disinfection action, and an optimum order and an optimum route of disinfection by a worker are indicated.

In the system 100 according to the present exemplary embodiments, the determination unit 101 and the storage unit 102 may be configured to communicate data (information) with each other via the communication units (104, 105) as illustrated in FIG. 1. With this configuration, the determination result that a disinfection action has been performed in the disinfection target areas and the time and location information about the performed disinfection action are transmitted by the terminal communication unit 104 of the terminal apparatus 110. The transmitted data is received by the server communication unit 105 of the server apparatus 120. The received data is stored in the storage unit 102.

### Detection Unit

The detection unit 103 according to the present exemplary embodiments is not particularly limited and may be any detection unit capable of detecting environmental change in the disinfection target areas. At least one unit selected from the group consisting of a unit configured to detect optical change, a unit configured to detect vibration, a unit configured to detect electrical change, and a unit configured to detect temperature change may be used.

### Unit Configured to Detect Optical Change

The unit configured to detect optical change according to the present exemplary embodiments detects change in light acquired from the disinfection target areas. Change in light herein refers to at least one of a change in light intensity and a change in light wavelength.

The unit configured to detect optical change according to the present exemplary embodiments may be an imaging unit such as a camera, a video camera, a terminal with a camera function, a smartphone with a camera function, a tablet with a camera function, or a monitoring camera. It is also possible to use a camera (wearable camera, mobile camera) that a worker who performs a disinfection action wears. A typical example of the unit configured to detect optical change is an image information acquisition unit, and either one of an image information acquisition unit that acquires still images and an image information acquisition unit that acquires moving images may be used.

In a case where the image information acquisition unit is configured to acquire moving images, a unit configured to acquire images of the disinfection target areas from moving images acquired during a predetermined period may be included. A unit configured to detect changed points in moving images to acquire images of the disinfection target areas from the moving images may be included. In this case, from the point of view of security, it is desirable to not store images other than the images of the disinfection target areas.

The unit configured to detect optical change may detect optical change in the disinfection target areas without acquiring images. For example, in a case where the disinfectant is liquid, whether the liquid has been applied may be detected using a liquid intrusion indicator. In a case where the disinfectant visualization sheet 403 described below is used, for example, an exhibited color (specific wavelength) of a visualization agent in the disinfection target areas may be detected. Alternatively, in a case where the visualization agent is in the form of a two-dimensional code or is placed to indicate text information or image information (diagram) as described below, the two-dimensional code, the text information, or the image information may be detected.

### Unit Configured to Detect Vibration

The unit configured to detect vibration according to the present exemplary embodiments may be any unit capable of detecting a vibration caused by a disinfection action, such as a vibration of equipment such as a table or a chair. For example, at least one of an acceleration sensor and a velocity sensor may be used to detect a vibration that indicates that a disinfection action has been performed. Either one of a contact-type acceleration sensor and a non-contact-type acceleration sensor may be used. Contact-type sensors such as piezoelectric sensors, conductive sensors, and strain gauge sensors may be used. Non-contact-type sensors such as eddy current sensors and displacement detection sensors such as capacitive sensors and optical sensors may be used.

### Unit Configured to Detect Electrical Change

The unit configured to detect electrical change according to the present exemplary embodiments detects electrical change in current or voltage caused by application of the disinfectant. Examples of the unit include pH meters, alcohol sensors, and circuits with current and voltage that are changed by application of a liquid.

### Unit Configured to Detect Temperature Change

The unit configured to detect temperature change according to the present exemplary embodiments detects temperature change in the disinfection target areas or areas near the disinfection target areas. For example, a temperature decrease caused by vaporization heat after application of the disinfectant such as alcohol to the disinfection target areas may be detected, or a temperature increase in a disinfection target item while a person is wiping the disinfectant applied to the disinfection target item may be detected. A publicly-known temperature sensor may be used as the unit configured to detect temperature change according to the present exemplary embodiments.

### Determination Unit

The determination unit 101 according to the present exemplary embodiments determines whether a disinfection action has been performed. A case where a disinfection action has been performed according to the present exemplary embodiments refers to, for example, a case where a disinfectant is applied to a disinfection target area and the applied disinfectant is wiped or a case where both the applying and the wiping are performed.

The application of the disinfectant and the wiping of the applied disinfectant may be performed by a person or by a machine.

### Storage Unit

The storage unit 102 according to the present exemplary embodiments stores a determination result that a disinfection action has been performed in the disinfection target areas, location information about the disinfection target areas, and time information about the performed disinfection action in association with each other. Not only the information indicating that a disinfection action has been performed but also the time and location information about the performed disinfection action are acquired, which makes it possible to perform disinfection efficiently in the plurality of disinfection target areas.

The storage unit 102 according to the present exemplary embodiments may use a storage medium such as a flexible disk, a hard disk, an optical disk, a magneto-optical disk, a CD-ROM, a CD-R, a magnetic tape, a non-volatile memory card, or a DVD besides a memory such as a ROM or a RAM

### Time Information About Performed Disinfection Action

The time information about the performed disinfection action according to the present exemplary embodiments is not particularly limited and may be any information indicating the time at which the disinfection action has been performed or equivalent information. At least one selected from the group consisting of a time at which the disinfectant has been wiped, a time at which the wiping of the disinfectant has been checked, and an elapsed time from when a previous disinfection action has been performed is used.

### Location Information About Performed Disinfection Action

The location information about the performed disinfection action according to the present exemplary embodiments may be a position at which a disinfection action target item is placed or a room in which a disinfection action target item is placed.

The location information about the performed disinfection action according to the present exemplary embodiments is not particularly limited and may be any information that enables identification of the position at which the disinfection action has been performed. Examples the location information include a target item on which the disinfection action has been performed, a position, an area, and a target item. Examples of the target item include spaces for shared use by an unspecified number of people such as conference rooms, coworking spaces, manga cafes, public facilities, and chairs, tables, and restrooms in the spaces.

### Disinfectant

The disinfectant according to the present exemplary embodiments is not particularly limited but is typically a liquid composition that is sprayed to the disinfection target areas. Examples of active ingredients of liquid compositions include surfactants, alkaline components, acidic components, and alcohols. Examples of alkaline components include alkali, sodium hypochlorite, hypochlorous acid, and sodium salts thereof.

### Analysis Unit

The analysis unit 106 performs analysis based on the determination result, the location information about the performed disinfection action, and the time information about the performed disinfection action that are stored in the storage unit 102. For example, in disinfection actions in the plurality of disinfection target areas, the analysis unit 106 is capable of outputting recommendation information about a recommended disinfection order. Examples of recommendation information include an order that leads to the fastest disinfection action completion.

### Terminal Communication Unit, Server Communication Unit

The terminal communication unit 104 and the server communication unit 105 according to the present exemplary embodiments communicate information (data) such as determination results. For example, a LAN may be used, which may be either one of a wired LAN and a wireless LAN, or a WAN may be used. The terminal communication unit 104 is realized by, for example, a wireless communication module and controls communication with the server communication unit 105 and communication networks. The same applies to the server communication unit 105.

### Terminal Apparatus

An example of the terminal apparatus 110 according to the present exemplary embodiments includes the detection unit 103, the determination unit 101, and the terminal communication unit 104 as illustrated in FIG. 1. The terminal apparatus 110 according to the present exemplary embodiments transmits, to the server apparatus 120, at least a determination result that a disinfection action has been performed, in a case where a disinfection action has been performed.

The terminal apparatus 110 according to the present exemplary embodiments may be a dedicated terminal for the disinfection action determination or a general-purpose smartphone or tablet.

### Server Apparatus

The server apparatus 120 according to the present exemplary embodiments communicates information (data) with the terminal apparatus 110 in the disinfection target areas and has functions such as storage and analysis functions.

An example of the server apparatus 120 will be described below with reference to the schematic diagram in FIG. 3. The server apparatus 120 includes the communication unit 301 (server communication unit 105), the storage unit 302 (storage unit 102), the operation unit 303, the display unit 304, the ROM 305, the RAM 306, and the control unit 310. The communication unit 301 is realized by, for example, a LAN card and controls communication with external apparatuses (e.g., terminal apparatus 110) and communication networks. The ROM 305 is realized by, for example, a non-volatile memory and stores various programs. The RAM 306 is realized by, for example, a volatile memory and temporarily stores various information. The storage unit 302 is realized by, for example, a HDD, a SSD, or a NAS and stores various information. The operation unit 303 is realized by, for example, a keyboard and/or a mouse, and the display unit 304 is realized by, for example, a display and displays various information to users.

The control unit 310 includes the data recording/updating unit 307 and the data processing unit 308. The data recording/updating unit 307 records the determination result that a disinfection action has been performed, time information about the performed disinfection action, and location information about the performed disinfection action in the storage unit 302, the ROM 305, and/or the RAM 306. The data processing unit 308 refers to, for example, databases stored in the storage unit 302, the ROM 305, and/or the RAM 306, adds information to read information, converts read information, and/or performs data processing on read information. The processing results may be communicated to the terminal apparatus 110 using the communication unit 301.

### Modified Examples

The system 100 according to another aspect of the present exemplary embodiments includes the detection unit 103 configured to detect environmental change in the disinfection target areas and output information about the environmental change and means (104) for transmitting information about whether a disinfection action has been performed in the disinfection target areas based on the information about the environmental change. In the example, the information about the environmental change in the disinfection target areas, e.g., a numerical value, which is output from the detection unit 103 is output, and in a case where the output information is greater than the predetermined threshold, information indicating that a disinfection action has been performed is transmitted.

### Method

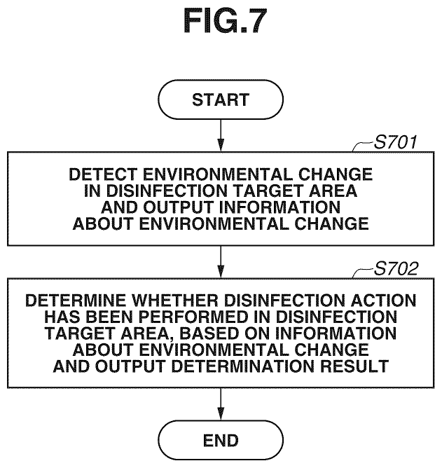

A method according to the present exemplary embodiments includes at least the following processes as illustrated in FIG. 7.
(1) A process of detecting environmental change in the disinfection target areas and outputting information about the environmental change (step S701).
(2) A process of determining whether a disinfection action has been performed in the disinfection target areas, based on the information about the environmental change, and outputting the determination result (step S702).

### First Modified Example of Exemplary Embodiments

A system according to a first modified example of the present exemplary embodiments will be described below with reference to FIG. 1. A case where the detection unit 103 according to the present modified example is a monitoring camera capable of detecting environmental change in the disinfection target areas will be described below as an example.

According to the present modified example, a camera 103 is used as the detection unit 103 to detect environmental change, i.e., a disinfection action has been performed, in the disinfection target areas. In this case, it is determined that a disinfection action has been performed in a case where an image characteristic of a disinfection action is acquired. For example, in a case where a worker performing disinfection is determined as wiping the disinfectant, it is determined that a disinfection action has been performed. The determination result that a disinfection action has been performed is transmitted to the server apparatus 120 together with time information about the time determined as the time at which the disinfection action has been performed, and location information about the performed disinfection action. The storage unit 102 of the server apparatus 120 stores the determination result, the time information about the time determined as the time at which the disinfection action has been performed, and the location information about the performed disinfection action in association with each other.

The storing in the storage unit 102 is performed similarly for the plurality of disinfection target areas.

The analysis unit 106 provides information (recommendation information) about a recommended order of disinfection in the plurality of disinfection target areas based on the information stored in the storage unit 102.

In the present modified example, the monitoring camera is used, so that no new devices for collecting information for the disinfection action determination are necessary.

### Second Modified Example of Exemplary Embodiments

A system according to a second modified example of the exemplary embodiments uses the disinfectant visualization sheet 403 in the determination of whether a disinfection action has been performed, in the system according to the first exemplary embodiment. According to the present modified example, application of the disinfectant to the disinfectant visualization sheet 403 visualizes the application on the disinfectant visualization sheet 403.

The disinfectant visualization sheet 403 will be described specifically below.

### Disinfectant Visualization Sheet

The disinfectant visualization sheet 403 according to the present modified example includes the substrate 402 and the color reaction portion 401 disposed on the substrate 402 as illustrated in FIG. 4A. The color reaction portion 401 includes the visualization agent (not illustrated), and application of the disinfectant to the visualization agent changes a color reaction state of the visualization agent. Use of the disinfectant visualization sheet 403 according to the present modified example as described above makes it possible to check whether the disinfectant has been applied, by visually checking a change in the color reaction state, which is convenient. The situation in which the disinfectant and the visualization agent are mixed together is when the disinfectant is applied to the disinfectant visualization sheet 403, and the time during which the disinfectant and the visualization agent are mixed together is short. This improves long-lasting preservation of materials that are used.

### Color Reaction Portion

The color reaction portion 401 according to the present modified example includes at least the visualization agent. The color reaction portion 401 may include a binder to enhance adhesion with the substrate 402. A resin may be used as the binder. For example, urethane resin or polyvinyl alcohol may be used.

The color reaction portion 401 desirably has a thickness of 1 μm or more and 80 μm or less, more desirably 5 μm or more and 60 μm or less, particularly desirably 15 μm or more and 50 μm or less. The color reaction portion 401 that is thicker than 80 μm is undesirable because adhesion with the substrate 402 decreases. The color reaction portion 401 that is thinner than 1 μm is undesirable because sufficient color reaction cannot be achieved.

### Visualization Agent

The visualization agent according to the present modified example is not particularly limited and may be any visualization agent that reacts with the disinfectant to cause a change in the color reaction state. A change in the color reaction state according to the present modified example refers to a change in color that is visually detectable before and after application of the disinfectant. Examples of the change include an occurrence of at least one of a change from a visually undetectable state to a visually detectable state, a change from a visually detectable state to a visually undetectable state, and a change in color that is visually detectable. A change in color that is visually detectable herein includes a change in color shade.

A phenomenon of change in the color reaction state is chromism. Chromism includes photochromism, thermochromism, electrochromism, acidochromism, solvatochromism, and vaporochromism. According to the present modified example, substances that cause the phenomena are referred to as chromic substances, particularly as photochromic substances, thermochromic substances, electrochromic substances, acidochromic substances, solvatochromic substances, and vaporochromic substances.

Acidochromic substances change color due to a pH change, and the color range of each substance varies depending on properties of the substance. An example of the substance is at least one type selected from the group consisting of Metanil Yellow, Metacresol Purple, Thymol Blue, Tropaeolin O, 2,4-dinitrophenol, Methyl Yellow, Bromophenol Blue, Congo Red, Methyl Orange, Bromochlorophenol Blue, Alizarin Red S, Bromocresol Green, Methyl Orange-Xylene Cyanol FF, 2,5-dinitrophenol, Methyl Orange-Indigo Carmine, Methyl Red, Methyl Orange-Xylene Cyanol FF-phenolphthalein, lacmoid, Chlorophenol Red, o-nitrophenol, p-nitrophenol, Bromocresol Green-Methyl Red, Bromocresol Purple, Bromophenol Red, Methyl Red-Methylene Blue, Bromothymol Blue, Neutral Red, Phenol Red, Neutral Red-Bromothymol Blue, Cresol Red, alpha-naphtholphthalein, Bromothymol Blue-Phenol Red, curcumin, phenolphthalein, Cresol Red-Thymol Blue, o-Cresolphthalein, alpha-Naphtholbenzein, Thymolphthalein, Thymol Blue-phenolphthalein, Alizarin Yellow GG, Alizarin Yellow R, Tropaeolin O, nitramine, 1,3,5-trinitrobenzene, Indigo Carmine, Methyl Violet, litmus, and Methyl Purple. There are substances that change color in one of acidic and alkaline pH ranges, while there are other substances that change color in both acidic and alkaline pH ranges, such as Thymol Blue. Substances that have a low level of toxicity and are more desirable are Metanil Yellow, Metacresol Purple, Thymol Blue, Tropaeolin O, Bromophenol Blue, Bromochlorophenol Blue, Alizarin Red S, Bromocresol Green, Methyl Red, lacmoid, Chlorophenol Red, o-Nitrophenol, Bromocresol Purple, Bromophenol Red, Bromothymol Blue, Neutral Red, Phenol Red, Cresol Red, alpha-Naphtholphthalein, phenolphthalein, o-Cresolphthalein, thymolphthalein, Alizarin Yellow GG, Alizarin Yellow R, Tropaeolin O, Methyl Violet, litmus, and Methyl Purple. Substances that are more desirable due to the reason of change color at a pH from 3 to 11 inclusive are Metacresol Purple, Thymol Blue, Tropaeolin O, Bromophenol Blue, Bromochlorophenol Blue, Alizarin Red S, Bromocresol Green, Methyl Red, lacmoid, Chlorophenol Red, o-Nitrophenol, Bromocresol Purple, Bromophenol Red, Bromothymol Blue, Neutral Red, Phenol Red, Cresol Red, alpha-Naphtholphthalein, phenolphthalein, o-Cresolphthalein, thymolphthalein, Alizarin Yellow GG, Alizarin Yellow R, litmus, and Methyl Purple. Desirably, alkaline disinfectants change color in alkaline conditions, and acidic disinfectants change color in acidic conditions.

Further, as the disinfectant evaporates, the visualization agent changes color and is restored. The restoration herein refers to the returning to a former state before the application of the disinfectant. In other words, the color reaction state is reversible. Specifically, the visualization agent according to the present modified example that changes its color reaction state for a predetermined time upon application of the disinfectant and thereafter returns to the former state before the application of the disinfectant may be used. For example, the color reaction state of the visualization agent in a visually undetectable state changes to a visually detectable state upon application of the disinfectant, and after the predetermined time passes, the color reaction state returns to the visually undetectable state. Being reversible as described above, the visualization agent may be used in situations where disinfection needs to be performed repeatedly.

Two or more types of visualization agents may be used. For example, the use of visualization agents that maintain their displayed colors for different durations from each other is desirable because this makes it possible to display a time from the contact with the disinfectant as an indicator.

### Substrate

The substrate 402 according to the present modified example may be any substrate on which the color reaction portion 401 is able to be disposed. The substrate 402 that is a resin sheet is suitable because it is lightweight and flexible. Examples of resin sheets include polyester resins such as polyethylene terephthalate, polybutylene terephthalate, and polyethylene terephthalate/isophthalate copolymers, polyolefin resins such as polyethylene, polypropylene, and polymethylpentene, polyethylene fluoride resins such as polyvinyl fluoride, polyvinylidene fluoride, polytetrafluoroethylene, and ethylene-tetrafluoroethylene copolymers, aliphatic polyamide resins such as Nylon 6 and Nylon 6,6, vinyl polymer resins such as polyvinyl chloride, vinyl chloride/vinyl acetate copolymers, ethylene/vinyl acetate copolymers, ethylene/vinyl alcohol copolymers, polyvinyl alcohol, and vinylon, cellulose-based resins such as triacetate cellulose and cellophane, acrylic resins such as polymethyl methacrylate, polyethyl methacrylate, polyethyl acrylate, and polybutyl acrylate, and other synthetic resins such as polystyrene, polycarbonate, polyarylate, and polyimide. One type of a resin sheet may be used alone, or two or more types of resin sheets may be combined or layered and used. Desirably, the substrate 402 is provided with a reception layer for retaining the color reaction portion 401 because in a case where the visualization agent is ink, it is easy for the reception layer to retain the visualization agent.

Desirably, the reception layer of the substrate 402 according to the present modified example does not react with the visualization agent. For example, in a case where the visualization agent displays color in alkaline conditions, the reception layer is desirably neutral or acidic. The reception layer that reacts with the visualization agent is undesirable because which causes the visualization agent to exhibit a color reaction prior to a reaction with the disinfectant.

The substrate 402 according to the present modified example may be transparent or opaque or may have a color. In a case where the visualization agent has a color before reacting with the disinfectant, the substrate 402 desirably has a color close to the color of the visualization agent.

Release paper, metal sheet, or wood may be used as the substrate 402 according to the present modified example. An adhesive portion may be provided to bond the substrate 402 to another member.

### Retention Portion

The disinfectant visualization sheet 403 according to the present modified example may include the retention portion 404 on the color reaction portion 401 to retain the disinfectant as illustrated in FIG. 4B. The retention portion 404 makes it possible to extend the time during which the disinfectant is retained, and this makes it possible to extend the time during which the disinfectant reacts with the visualization agent and color is exhibited. Further, the color reaction time is controllable by suitably selecting materials to be provided to the retention portion 404. For example, the time of changing from the visually detectable color reaction state to the visually undetectable state is controlled.

Having the foregoing function, the retention portion 404 may also be referred to as fading time control portion.

Desirably, the retention portion 404 includes empty spaces to retain the applied disinfectant. For example, the retention portion 404 may be formed using a resin and inorganic particles that form empty spaces. The inorganic particles for use herein are of at least one type selected from the group consisting of pearl necklace-shaped silica particles, chain-like silica particles, spherical colloidal silica, non-spherical colloidal silica, alumina particles, titania particles, and zirconia particles. Desirably, a peak position of a particle size distribution of particle diameters of inorganic fine particles analyzed by dynamic light scattering is at 1 nm or more and 300 nm or less, more desirably 1 nm or more and 150 nm or less, particularly desirably 1 nm or more and 60 nm or less. Especially for pearl necklace-shaped silica particles, the peak position is desirably at 20 nm or more and 50 nm or less. As for chain-like silica particles, the peak position is desirably at 1 nm or more and 10 nm or less. Large particle diameters are undesirable because empty spaces decrease. Particle diameters that are excessively small are also undesirable because pore diameters of empty spaces formed by inorganic particles become so small that the applied disinfectant is absorbed at low speed. Desirably, a pore diameter distribution curve of the pore diameters has a maximum peak in the range of 5 nm or more and 20 nm or less inclusive.

With a peak of pore diameters at 5 nm or less, the absorption rate of the disinfectant becomes slow, whereas with a peak beyond 20 nm, haze may increase. In a case where haze increases, whitishness becomes noticeable, and the visibility of the color reaction portion 401 decreases. Thus, this is undesirable. Pore diameters can be measured by BET specific surface area measurement.

While resins for use herein may be selected from various resins, in a case where the disinfectant is a water-based disinfectant, a water-soluble resin is desirable. For example, at least one type of a water-soluble resin selected from the group consisting of cellulose-based binders such as methylcellulose, methylhydroxyethyl cellulose, methylhydroxypropyl cellulose, and hydroxyethyl cellulose, starch and modified products thereof, gelatin and modified products thereof, natural polymer resins such as casein, pullulan, Arabic gum, karaya gum, and albumin or derivatives thereof, polyvinyl alcohol and modified products thereof, latex and emulsions such as styrene-butadiene rubber (SBR) latex, nitrile butadiene rubber (NBR) latex, methyl methacrylate-butadiene copolymers, and ethylene-vinyl acetate copolymers, polyacrylamide, vinyl polymers such as polyvinylpyrrolidone, polyethyleneimine, polypropylene glycol, polyethylene glycol, and maleic anhydride or copolymers thereof may be used.

The thickness of the retention portion 404 does not need to be uniform. For example, the disinfectant retention time is controllable by changing the thickness of a portion. This is desirable because such a configuration makes it possible to control the color reaction time of the color reaction portion 401, and the time from the contact with the disinfectant can be displayed as an indicator.

### Protection Portion

According to the present modified example, a protection portion for protecting the color reaction portion 401 may be provided on the color reaction portion 401. The phrase “on the color reaction portion 401” herein indicates that the protection portion may be provided in contact with the color reaction portion 401 or, in a case where the retention portion 404 is provided on the color reaction portion 401, the protection portion may be provided on the retention portion 404. For example, in a case where a water-soluble resin is used as the retention portion 404, since a surface becomes viscous, it is desirable to coat with the protection layer. At least one type selected from the group consisting of resins such as acrylic resins, vinyl acetate resins, vinyl chloride resins, ethylene/vinyl acetate copolymer resins, polyamide resins, polyester resins, urethane resins, polyolefin resins, and copolymer resins thereof may be used as the protection portion according to the present modified example.

Since the surface of the disinfectant visualization sheet 403 is wiped with the disinfectant repeatedly, the fading time control portion is desirable because it provides durability.

### Two-Dimensional Code

The disinfectant visualization sheet 403 according to the present modified example may include the visualization agent in the shape of a two-dimensional code.

The two-dimensional code is at least one two-dimensional code selected from the group consisting of Quick Response code (QR code®), micro QR code, SP code, VeriCode, MaxiCode, CP code, DataMatrix, DataMatrix ECC200, Code1, AztecCode, INTACTA.CODE, card e, Chameleon Code, PDF417, micro PDF417, Code49, Code16K, Codablock, SuperCode, UltraCode, RSS Composite, and AztecMesa. With the visualization agent being provided to form a two-dimensional code on the substrate 402, the imaging unit reads the two-dimensional code, and the determination of whether the disinfectant has been applied is performed based on the image information.

From the two-dimensional code, information about the time of application of the disinfectant and information about the location of application of the disinfectant are obtainable. Upon reading the two-dimensional code, text information or image information indicating completion of disinfection (cleaning) may be displayed on a display unit of a terminal having read the two-dimensional code. In a case where a disinfection target area is a place that requires payment of a fee, such as a store or a coworking space, reading the two-dimensional code may connect to an attached fee payment system. Upon reading the two-dimensional code, information such as text information indicating that points that can be used at the store are provided or returned, that a discount is granted, or a gift is presented may be displayed to motivate the user to perform disinfection (cleaning).

### Another Example of Disinfectant Visualization Sheet

In another example of the disinfectant visualization sheet 403 according to the present modified example, the color reaction portion 401 may be provided with visualization particles including the visualization agent and carrier particles carrying the visualization agent. An agglomerate of visualization particles is configured to function as the color reaction portion 401. In a case where the visualization particles are used, positions of a hydration layer and a layer including the visualization particles can be designed microscopically, which enables precise characteristic design.

### Invisible Portion

In yet another example of the disinfectant visualization sheet 403 according to the present modified example, a combination of a plurality of principles may be used in addition to the visualization agent. A sheet including the above-described visualization color reaction portion may be provided with an invisible portion. The invisible portion according to the present modified example may be provided with invisible materials that absorb light with ultraviolet and infrared wavelengths. The ultraviolet and infrared ranges include near-ultraviolet and near-infrared ranges. With the invisible agent, additional checking from a third-party perspective becomes possible in addition to visual checking of whether the disinfectant has been applied.

Furthermore, the disinfectant visualization sheet 403 according to the present modified example may be used in further combination with thermochromism. For example, the visualization color reaction portion is used in combination with a dye that changes color in response to temperature. Consequently, in a case where there is a change in temperature from a use environment, the exhibited color further changes in addition to the color change based on the presence or absence of the disinfectant. This leads to recognition of a touch history indirectly, for example. A thermochromism dye may be selected from publicly-known substances.

As a dye that absorbs the infrared spectrum among the invisible materials according to the present modified example, at least one type selected from the group consisting of phthalocyanine dyes, naphthalocyanine dyes, metal complex dyes, polymethine dyes, quinone dyes, azo dyes, diphenylmethane and triphenylmethane dyes, radical dyes, perimidine dyes, and Au nanorods may be used. As a dye that absorbs the ultraviolet spectrum and emits fluorescence, at least one type selected from the group consisting of melocyanin, perylene, acridine, luciferin, pyranine, stilbene, rhodamine, coumarin, and fluorescein may be used.

### Visually Detectable Wavelength Range

A visually detectable wavelength range according to the present modified example refers to light in the wavelength range from 360 nm or more to 830 nm or less inclusive. The infrared wavelength range is from 900 nm or more to 14 μm or less inclusive, and the ultraviolet wavelength range is from 100 nm or more to 400 nm or less inclusive.

### Marking Composition

The color reaction portion 401 of the disinfectant visualization sheet 403 according to the present modified example may be provided as a marking indicating whether the disinfectant has been applied (cleaned). A material for marking according to the present modified example may be referred to as marking composition. The marking composition contains the visualization agent and a solvent, and the solvent may be a hydrophobic solvent or an aqueous solvent. The hydrophobic solvent may be an organic solvent such as heptane or petroleum ether, and the aqueous solvent may be water or alcohol.

As the aqueous solvent according to the present modified example, water or a mixed medium using water as a major solvent in combination with a protonic organic solvent or a non-protonic organic solvent may be used. It is desirable to use a medium that mixes with or dissolves in water at any proportion, desirably a uniform mixed medium containing 50% or more by mass of water, as the organic solvent according to the present modified example. As the water, deionized water (ion-exchanged water) or ultrapure water is desirably used.

The protonic organic solvent is an organic solvent containing hydrogen atoms (acidic hydrogen atoms) bonded to oxygen or nitrogen. The non-protonic organic solvent is an organic solvent without an acidic hydrogen atom. Examples of organic solvents include alcohols, alkylene glycols, polyalkylene glycols, glycol ethers, glycol ether esters, carboxylic acid amides, ketones, keto alcohols, or cyclic ethers.

Examples of aqueous mediums that are suitable for use include water, water/ethanol mixed solvents, water/ethylene glycol mixed solvents, or water/N-methylpyrrolidone mixed solvents. The water content based on the total mass of the composition is desirably 10.0% or more by mass and 90.0% or less by mass, desirably 50.0% or more by mass and 90.0% or less by mass.

The content of the water-soluble organic solvent in the composition based on the total mass of the composition is desirably 5.0% or more by mass and 90.0% or less by mass, more desirably 10.0% or more by mass and 50.0% or less by mass.

### Other Additives

The marking composition according to the present modified example may further contain, in addition to the above-described components as needed, a water-soluble organic compound such as polyhydric alcohols such as trimethylolpropane and trimethylolethane, urea, or urea derivatives such as urea and ethylene urea. Furthermore, the marking composition according to the present modified example may contain various additives as needed such as a surfactant, a pH adjuster, a rust inhibitor, an anticorrosive agent, an antimicrobial agent, an antioxidant, a reduction inhibitor, an evaporation enhancer, a chelating agent, and a water-soluble resin. The surfactant may be an anionic, cationic, or nonionic surfactant. The content of the surfactant in the marking composition based on the total mass of the composition is desirably 0.1% or more by mass and 5.0% or less by mass, more desirably 0.1% or more by mass and 2.0% or less by mass. Specific examples of surfactants that are suitable for use include nonionic surfactants such as polyoxyethylene alkyl ether, polyoxyethylene fatty acid ester, polyoxyethylene alkyl phenyl ether, polyoxyethylene-polyoxypropylene block copolymers, and acetyleneglycol-based compounds.

In recording images on recording mediums by discharging the marking composition according to the present modified example from an inkjet recording head, it is desirable to use the marking composition that has an appropriately-controlled surface tension and an appropriately-controlled viscosity. Specifically, the density of colorant compounds in the marking composition is desirably about 5% to about 20%. The surface tension of the composition at 25° C. is desirably 10 mN/m or more and 60 mN/m or less, more desirably 20 mN/m or more and 60 mN/m or less, particularly desirably 30 mN/m or more and 50 mN/m or less. The viscosity of the composition at 25° C. is desirably 1.0 mPa·s or more and 10 mPa·s, more desirably 1.0 mPa·s or more and 5 mPa·s or less.

### Image Recording Method [Marking Composition Application Method]

An image recording method according to the present modified example relates to a method for applying the marking composition to a substrate. Various methods may be used in the image recording method according to the present modified example, and examples thereof include inkjet methods, flexographic method, screen methods, offset methods, and spin coating methods. For example, inkjet methods are methods for recording images on recording mediums by discharging the marking composition according to the present modified example from an inkjet recording head. A method for discharging the marking composition may be a method in which mechanical energy is applied to the marking composition and a method in which thermal energy is applied to the composition. Publicly-known methods may be used in the processes of the inkjet recording method, except that the marking composition according to the present modified example is used.

### Disinfectant Visualization Particles

The color reaction state of the disinfectant visualization particles 405 according to the present modified example changes upon application of the disinfectant. Specifically, the disinfectant visualization particles 405 each include a visualization agent 420 and a carrier particle 410 carrying the visualization agent 420 as illustrated in FIG. 4C. The color reaction state of the visualization agent 420 changes upon application of the disinfectant. The disinfectant visualization particles 405 according to the present modified example may include the fading time control portion (not illustrated). The fading time control portion does not necessarily need to be layered. Porous particles may be used as the fading time control portion. For example, at least one type selected from the group consisting of mesoporous silica particles, porous silica particles, porous titania particles, porous zirconia particles, porous ceria particles, porous zinc oxide particles, porous crosslinked polymethyl methacrylate particles, porous crosslinked polystyrene particles, and porous methyl methacrylate-styrene copolymer crosslinked particles may be used. By impregnating the porous particles with a solution in which the visualization agent is dissolved and then drying the porous particles, the disinfectant visualization particles 405 including the color reaction portion 401 are formed.

The porous particles may be treated to have a hydrophilic or hydrophobic surface. In a case where the disinfectant is a water-based disinfectant, hydrophilic surfaces are desirable, whereas in a case where the disinfectant is not a water-based disinfectant, hydrophobic surfaces are desirable.

Since the disinfectant visualization particles 405 have high rigidity, the durability remains high even after wiping of the disinfectant is performed repeatedly, so that using the disinfectant visualization particles 405 is desirable.

The particle diameter of the disinfectant visualization particles 405 is desirably 30 nm or more and 5 μm or less, more desirably 50 nm or more and 3 μm or less, particularly desirably 80 nm or more and 1 μm or less. A particle diameter less than 30 nm is undesirable because dispersion efficiency is low, which raises an issue in long lasting stability. A particle diameter greater than 5 μm is undesirable because sedimentation often occurs in dispersing the disinfectant visualization particles 405 in a solution.

### Sheet Using Disinfectant Visualization Particles

The disinfectant visualization particles 405 may be provided on the substrate 402 as illustrated in FIG. 4D to use the disinfectant visualization particles 405 according to the present modified example as a sheet using the disinfectant visualization particles 405.

### Coating Liquid and Coated Article With Disinfectant Visualization Particles

The disinfectant visualization particles 405 according to the present modified example may be used as a coating liquid such as a dispersion liquid or a slurry. The coating liquid may be applied to an article to form a coated article.

### Modified Example of Disinfectant Visualization Particles

The disinfectant visualization particles 405 according to the present modified example do not necessarily need to be in the shape of particles. For example, a rod shape, a plate shape, or a film shape may be employed. In the case of a film shape, it is especially desirable to provide the fading time control portion in order to maintain strength against wiping of the disinfectant.

### Third Modified Example of Exemplary Embodiments

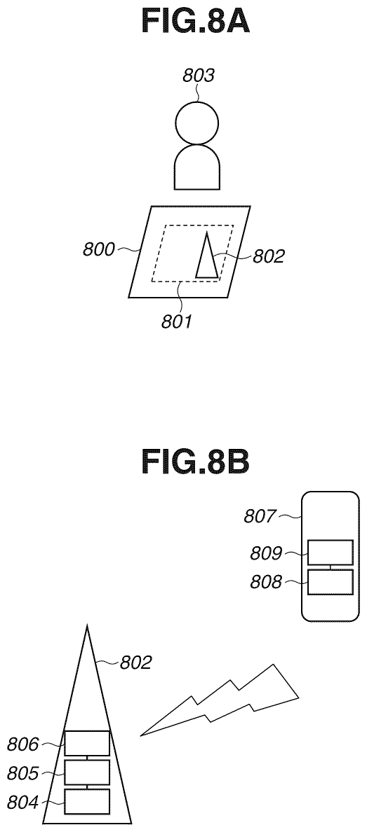

A system according to a third modified example of the exemplary embodiments uses means for detecting vibration as the detection unit 103. An example using an acceleration sensor will be described below with reference to FIGS. 8A and 8B.

The system according to the present modified example is configured to detect that a disinfection action has been performed on equipment 800 by a person (worker) 803. The equipment 800 includes a disinfection target area 801 and is provided with a terminal apparatus 802 for checking whether a disinfection action has been performed. The terminal apparatus 802 includes a detection unit (acceleration sensor) 804 and a determination unit 805. The detection unit 804 detects environmental change in the disinfection target area 801 (vibration of the equipment 800 in this example), and the determination unit 805 outputs, based on the vibration detection, a determination result about whether a disinfection action has been performed. A terminal transmission unit 806 configured to transmit determination results transmits data on the determination result to a server apparatus 807. A server communication unit 808 of the server apparatus 807 having received the data on the determination result stores the determination result in a storage unit 809.

As described above, the system according to the present modified example detects that a disinfection action has been performed without recording by the person 803 and stores the detection result in the storage unit 809 of the server apparatus 807. Thus, whether a disinfection action has been performed can be checked easily by referring to the data stored in the storage unit 809.

### Fourth Modified Example of Exemplary Embodiments

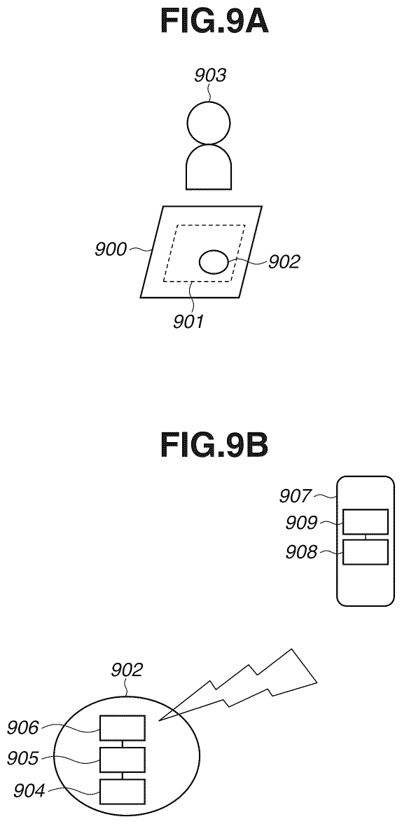

FIGS. 9A and 9B are diagrams illustrating a system according to a fourth modified example of the exemplary embodiments using an alcohol sensor as the detection unit 103.

The system according to the present modified example is configured to detect that a disinfection action has been performed on equipment 900 by a person (worker) 903. The equipment 900 includes a disinfection target area 901 and is provided with a terminal apparatus 902 for checking whether a disinfection action has been performed. The terminal apparatus 902 includes a detection unit (alcohol sensor) 904 and a determination unit 905. The detection unit 904 detects environmental change in the disinfection target area 901 (equipment 900 in this example), and the determination unit 905 outputs, based on the alcohol detection, a determination result about whether a disinfection action has been performed. A terminal transmission unit 906 configured to transmit environmental change detection results transmits data on the detection result to a server apparatus 907. Based on the detection result, a determination unit 908 of the server apparatus 907 having received the data on the detection result determines whether a disinfection action has been performed, and the determination result is stored in a storage unit 909.

As described above, the system according to the present modified example detects that a disinfection action has been performed without recording by the person 903 and stores the detection result in the storage unit 909 of the server apparatus 907. Thus, whether a disinfection action has been performed can be checked easily by referring to the data stored in the storage unit 909.

As described above, with the system according to the modified examples of the present exemplary embodiments, a system by which whether a disinfection action has been performed can be checked easily is provided.

The present invention is not limited to the above-described exemplary embodiments, and various modifications and variations can be made without departing from the spirit and scope of the present invention. Accordingly, the following claims are appended to disclose the scope of the present invention.

The system according to the exemplary embodiments of the present invention provide assistance to perform disinfection efficiently and thoroughly without omissions in actual situations.

While the present invention has been described with reference to the exemplary embodiments, it is to be understood that the invention is not limited to the disclosed exemplary embodiments. The scope of the following claims is to be accorded the broadest interpretation so as to encompass all such modifications and equivalent structures and functions.

1. A system for assisting in a disinfection action in a disinfection target area, the system comprising:
a determination unit configured to acquire information about a determination result about whether the disinfection action has been performed in the disinfection target area; and
a storage unit configured to store the determination result that the disinfection action has been performed in the disinfection target area, time information about the performed disinfection action, and location information about the performed disinfection action in association with each other.

2. The system according to claim 1, further comprising an analysis unit configured to perform analysis based on location information about a plurality of the disinfection target areas and the time information about the performed disinfection action in the plurality of the disinfection target areas and output recommendation information about a recommended order of the disinfection action in the plurality of the disinfection target areas.

3. The system according to claim 1, wherein the determination result is information indicating that a disinfectant has been applied to the disinfection target area and the applied disinfectant has been wiped.

4. The system according to claim 1, wherein the time information about the performed disinfection action is at least one selected from the group consisting of a time at which the disinfectant has been wiped, a time at which the wiping of the disinfectant has been checked, and an elapsed time since a previous disinfection action.

5. The system according to claim 1, wherein the location information about the performed disinfection action is at least one selected from the group consisting of a position at which a disinfection action target item is placed and a room in which the disinfection action target item is placed.

6. The system according to claim 1, further comprising a detection unit configured to detect environmental change in the disinfection target area and output information about the environmental change,
wherein the determination unit determines whether the disinfection action has been performed in the disinfection target area, based on the information about the environmental change, and outputs a determination result.

7. The system according to claim 1, wherein the detection unit is at least one selected from the group consisting of a unit configured to detect optical change, a unit configured to detect vibration, a unit configured to detect electrical change, and a unit configured to detect temperature change.

8. A method for assisting in a disinfection action in a plurality of disinfection target areas, the method comprising:
determining whether the disinfection action has been performed in the disinfection target areas and outputting a determination result; and
storing the determination result that the disinfection action has been performed in the disinfection target areas, location information about the performed disinfection action, and time information about the performed disinfection action in association with each other.

9. A system comprising:
a detection unit configured to detect environmental change in a disinfection target area and output information about the environmental change; and
a determination unit configured to determine whether a disinfection action has been performed in the disinfection target area, based on the information about the environmental change.

10. The system according to claim 9, wherein the detection unit is at least one selected from the group consisting of a unit configured to detect optical change, a unit configured to detect vibration, a unit configured to detect electrical change, and a unit configured to detect temperature change.

11. The system according to claim 10, wherein the unit configured to detect optical change includes an imaging unit.

12. The system according to claim 10, wherein the unit configured to detect vibration includes at least one of an acceleration sensor and a velocity sensor.

13. The system according to claim 10, wherein the unit configured to detect electrical change includes at least one of a pH meter and an alcohol sensor.

14. A system comprising:
a detection unit configured to detect environmental change in a disinfection target area and output information about the environmental change; and
a transmission unit configured to transmit information about whether a disinfection action has been performed in the disinfection target area, based on the information about the environmental change.

15. A method comprising:
detecting environmental change in a disinfection target area and outputting information about the environmental change; and
determining whether a disinfection action has been performed in the disinfection target area, based on the information about the environmental change.
